(19) United States
(12) Patent Application Publication
(10) Pub. No.: US 2026/0118456 A1
(43) Pub. Date: Apr. 30, 2026

(54) POWER SUPPLY ASSEMBLY OF GRADIENT COIL AND MAGNETIC RESONANCE IMAGING SYSTEM

(71) Applicant: SHENZHEN UNITED IMAGING RESEARCH INSTITUTE OF INNOVATIVE MEDICAL EQUIPMENT INNOVATION RESEARCH, Shenzhen, Guangdong (CN)

(72) Inventor: Kejia NIU, Shenzhen (CN)

(73) Assignee: SHENZHEN UNITED IMAGING RESEARCH INSTITUTE OF INNOVATIVE MEDICAL EQUIPMENT INNOVATION RESEARCH, Shenzhen, Guangdong (CN)

(21) Appl. No.: 19/117,024

(22) PCT Filed: Sep. 27, 2023
(86) PCT No.: PCT/CN2023/122184

§ 371 Date: Mar. 28, 2025

(30) Foreign Application Priority Data
Sep. 29, 2022 (CN) 202211195860.6

(51) Int. Cl. G01R 33/36; G01R 33/385

(52) U.S. Cl. CPC G01R 33/36; G01R 33/3856; G01R 33/3858

(57) ABSTRACT

A power supply assembly of a gradient coil are provided. The power supply assembly of the gradient coil may include a first insulation device, wherein the first insulation device is located within a scanning channel of a main magnet and connected to one end of the gradient coil, the first insulation device may be arranged with a first conductive unit, the conductive unit may be a rigid conductor, a first end of the first conductive unit may be electrically connected with the gradient coil of a magnetic resonance imaging system, and a second end of the first conductive unit may be electrically connected with a cable of the magnetic resonance imaging system.

### CROSS-REFERENCE TO RELATED APPLICATIONS

This application claims priority to the Chinese Patent Application No. 202211195860.6, filed on Sep. 29, 2022, the contents of which are hereby incorporated by reference.

### TECHNICAL FIELD

The present disclosure relates to a magnetic resonance (MR) technology field, in particular, relates to a power supply assembly of a gradient coil and a magnetic resonance imaging (MRI) system.

### BACKGROUND

In traditional magnetic resonance imaging systems, a length of a gradient coil is slightly smaller than a length of a main magnet along an axial direction of an imaging bore. A conductive pillar is embedded in the gradient coil, so that an end surface of the conductive pillar is aligned with an end surface of the main magnet. A cable with a wiring terminal is directly pressed onto the conductive pillar, thus achieving power supply to the gradient coil through the cable. However, with a development of magnetic resonance imaging technology, a high-field or even ultra-high field magnetic resonance imaging system have emerged. The volume of the main magnet in the MRI system is relatively large, and a length of the main magnet along the axial direction is much larger than a length of the gradient coil. Traditional electrical connection results in a portion of the cable being exposed to the magnetic field environment. When current is applied, the gradient coil vibrates at a high frequency, causing the cable to vibrate accordingly. At this time, the cable is subjected to strong and high-frequency alternating Lorentz forces. components used for electrically connecting the cable and the gradient coil can not be fixed, which causes excessive stress to the cable, resulting in fatigue fracture of the cable. Therefore, it is desirable to provide a power supply assembly of a gradient coil and a magnetic resonance imaging system that can achieve a stable electrical connection between the power supply assembly and the gradient coil and reduce risk of cable breakage.

### SUMMARY

An aspect of the present disclosure relates to a power supply assembly of a gradient coil, comprising: a first insulation device, wherein the first insulation device is located within a scanning channel of a main magnet of a magnetic resonance imaging (MRI) system, the first insulation device is arranged with a first conductive unit, the first conductive unit is a rigid conductor, a first end of the first conductive unit is electrically connected with the gradient coil of the system, and a second end of the first conductive unit is electrically connected with a cable of the MRI system.

A further aspect of the present disclosure relates to a magnetic resonance imaging (MRI) system, comprising: a main magnet; a gradient coil; a power supply assembly of the gradient coil; and a cable; the cable configured to supply power to the gradient coil through the power supply assembly of the gradient coil; wherein the power supply assembly of the gradient coil includes a first insulation device, the first insulation device is located within a scanning channel of the main magnet, the first insulation device is arranged with a first conductive unit, the first conductive unit is a rigid conductor, a first end of the first conductive unit is electrically connected with the gradient coil, and a second end of the first conductive unit is electrically connected with the cable.

A still further aspect of the present disclosure relates to a power supply assembly of a gradient coil, comprising: an electrode insulation device and a conductive pillar insulation device matched with a gradient coil of a magnetic resonance imaging (MRI) system; wherein the electrode insulation device is arranged with an electrode, the electrode insulation device is arranged with a conductive pillar, and the electrode is connected with the cable and the conductive pillar to supply power for the gradient coil.

### BRIEF DESCRIPTION OF THE DRAWINGS

The present disclosure is further describable in terms of exemplary embodiments. These exemplary embodiments are describable in detail with reference to the drawings. These embodiments are non-limiting exemplary embodiments, in which like reference numerals represent similar structures throughout the several views of the drawings, and wherein:

FIG. 1 is a schematic diagram illustrating a structure of a magnetic resonance imaging system according to some embodiments of the present disclosure;

FIG. 2 is a schematic diagram illustrating a front view of a magnetic resonance imaging system according to some embodiments of the present disclosure;

FIG. 3 is a schematic diagram illustrating a cross section view of a magnetic resonance imaging system according to some embodiments of the present disclosure;

FIG. 4 is a schematic diagram illustrating a structure of a first insulation device according to some embodiments of the present disclosure;

FIG. 5 is a schematic diagram illustrating a structure of a second insulation device according to some embodiments of the present disclosure;

FIG. 6 is a schematic diagram illustrating a structure of a cable according to some embodiments of the present disclosure;

FIG. 7 is a schematic diagram illustrating a structure of a second fixing component according to some embodiments of the present disclosure;

FIG. 8 is a schematic diagram illustrating a structure of a safety shield of a second insulation device according to some embodiments of the present disclosure;

FIG. 9 is a schematic diagram illustrating a structure of a power supply assembly of a gradient coil facing ground according to other embodiments of the present disclosure;

FIG. 10 is a schematic diagram illustrating a structure of a connecting component according to some embodiments of the present disclosure;

FIG. 11 is a schematic diagram illustrating a structure of an integrated forming device according to some embodiments of the present disclosure;

FIG. 12 is a schematic diagram illustrating a side view of an integrated forming device according to some embodiments of the present disclosure; and

FIG. 13 is a schematic diagram illustrating a structure of a first supporting component according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been describable at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assemblies of different levels in ascending order. However, the terms may be displaced by other expressions if they achieve the same purpose.

In the description of the present invention, it should be understood that the terms “center,” “vertical,” “horizontal,” “length,” “width,” “thickness,” “up,” “down,” “front,” “back,” “left,” “right,” “vertical,” “horizontal,” “top,” “bottom,” “inside,” “outside,” “clockwise,” “counterclockwise,” “axial,” “radial.” The orientation or positional relationship indicated by “circumferential direction” is based on the orientation or positional relationship shown in the attached drawings, and is only for the convenience of describing the present invention and simplifying the description, rather than indicating or implying that the device or element referred to must have a specific orientation, be constructed and operated in a specific orientation, and therefore cannot be understood as a limitation of the present invention.

In addition, the terms “first” and “second” are only used to describe the purpose and cannot be understood as indicating or implying relative importance or implying the quantity of technical features indicated. Therefore, features limited to “first” and “second” can explicitly or implicitly include at least one of these features. In the description of the present invention, “multiple” means at least two, such as two, three, etc., unless otherwise specifically defined.

In the present invention, unless otherwise specified and limited, the terms “installation,” “connection,” “fixation” and other terms should be broadly understood, for example, they can be fixed connections, detachable connections, or integrated; it can be a mechanical connection or an electrical connection; it can be directly connected or indirectly connected through an intermediate medium, which can be the internal connection between two components or the interaction relationship between two components, unless otherwise specified. For ordinary technical personnel in this field, the specific meanings of the above terms in the present invention can be understood based on specific circumstances.

In the present invention, unless otherwise specified and limited, the first feature may be in direct contact with the second feature “above” or “below,” or the first and second features may be in indirect contact through intermediate media. Moreover, the first feature is “above” the second feature, but the first feature is directly or diagonally above the second feature, or simply indicates that the first feature is horizontally higher than the second feature. The first feature “below” of the second feature can indicate that the first feature is directly or diagonally below the second feature, or simply indicates that the horizontal height of the first feature is less than that of the second feature.

It should be noted that when a component is referred to as “fixed to” or “set to” another component, it can be directly on another component or there can be a centered component. When a component is considered to be ‘connected’ to another component, it can be directly connected to another component or there may be both centering components present. The terms “vertical,” “horizontal,” “up,” “down,” “left,” “right,” and similar expressions used in this article are for illustrative purposes only and do not necessarily represent the only implementation method.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

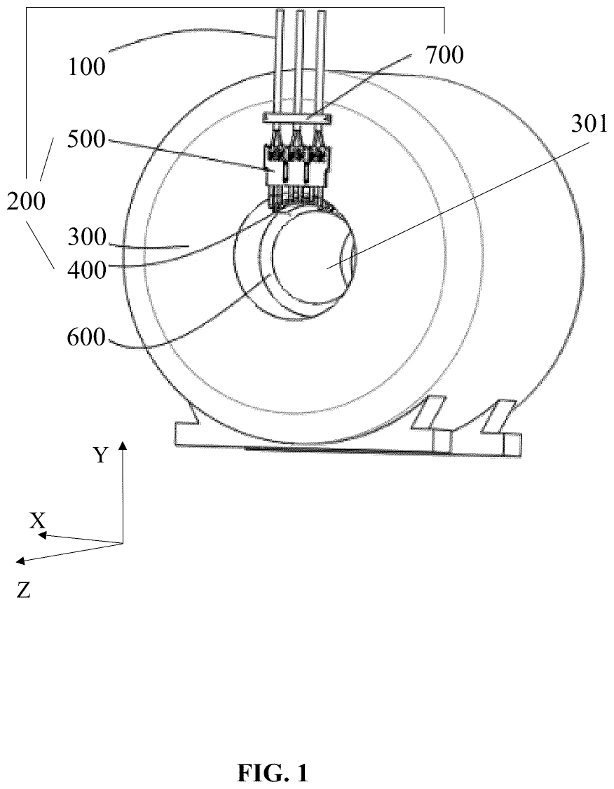

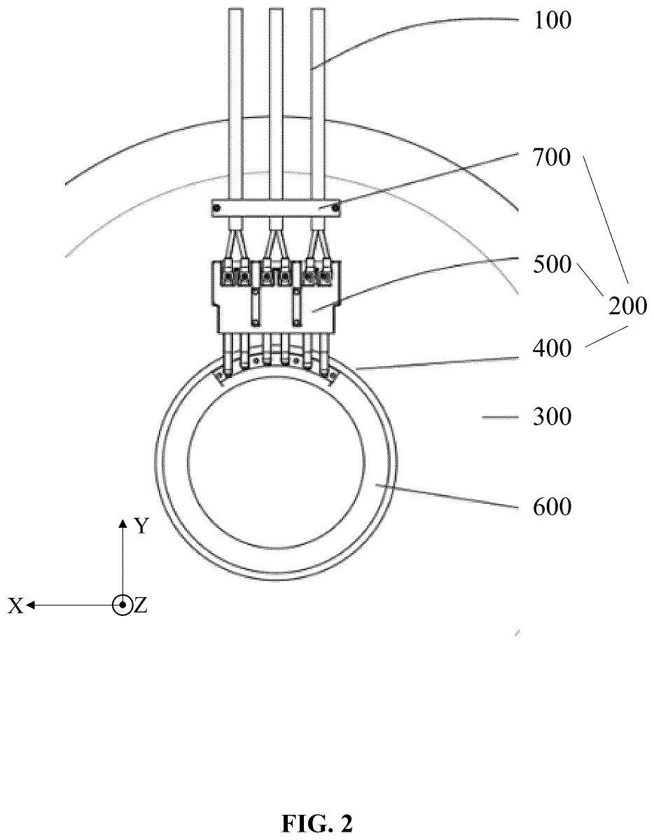

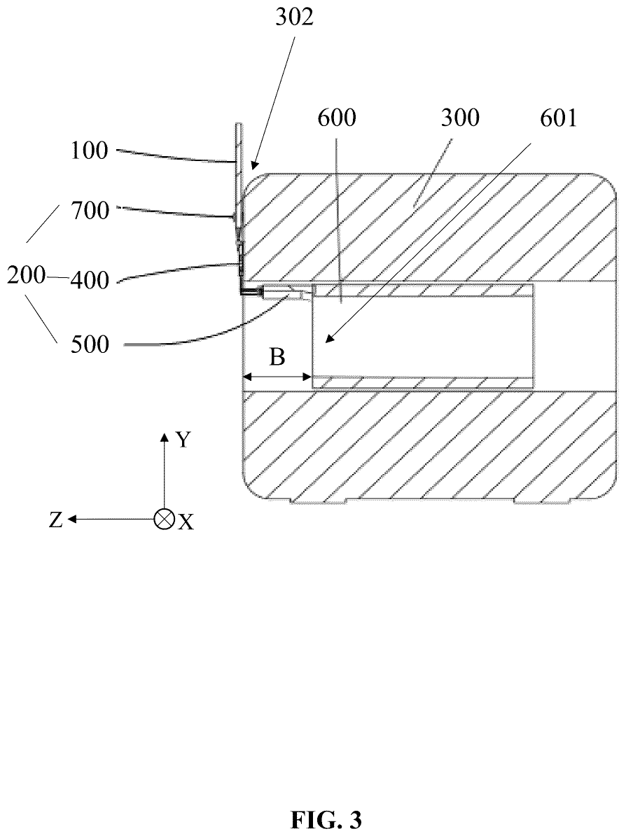

FIG. 1 is a schematic diagram illustrating a structure of an exemplary magnetic resonance imaging system according to some embodiments of the present disclosure. FIG. 2 is a schematic diagram illustrating a front view of an exemplary magnetic resonance imaging system according to some embodiments of the present disclosure. FIG. 3 is a schematic diagram illustrating a cross section view of an exemplary magnetic resonance imaging system according to some embodiments of the present disclosure. As shown in FIG. 1-FIG. 3. the magnetic resonance imaging system may include a cable 100, a power supply component 200, a main magnet 300, and a gradient coil 600.

The magnetic resonance imaging (MRI) system may scan an object located within its detection region and generate a plurality of data relating to the object. In the present disclosure, “subject” and “object” are used interchangeably. Mere by way of example, the object may include a patient, a man-made object, etc. As another example, the object may include a specific portion, organ, and/or tissue of a patient. For example, the object may include head, brain, neck, body, shoulder, arm, thorax, cardiac, stomach, blood vessel, soft tissue, knee, feet, or the like, or any combination thereof. In the present disclosure, the X axis, the Y axis, and the Z axis shown in FIG. 1 may form an orthogonal coordinate system. The X axis and the Z axis shown in FIG. 1 may be horizontal, and the Y axis may be vertical. As illustrated, the positive X direction along the X axis may be from the right side to the left side of the MRI system seen from the direction facing the front of the MRI system; the positive Y direction along the Y axis shown in FIG. 1 may be from the lower part to the upper part of the MRI system; the positive Z direction along the Z axis shown in FIG. 1 may refer to a direction in which the object is moved out of the scanning channel 301 (or referred to as a bore, a scanning bore, or an imaging bore) of the MRI system.

The cable 100 refers to a transmission device used for transmitting power and information, such as wires, or the like. The cable may be made of one or more mutually insulated conductors and an external insulation protective layer. The conductor may be made of metal materials such as copper, aluminum, and copper alloy, and the insulation protection layer may be made of polymer materials such as polyethylene and polyvinyl chloride. The cable 100 may be used to transmit current for the magnetic resonance imaging system (e.g., the gradient coil 600).

The main magnet 300 may generate a first magnetic field (or referred to as a main magnetic field) that may be applied to an object (also referred to as a subject) exposed inside the field. The main magnet 300 may include a resistive magnet or a superconductive magnet that both need a power supply (not shown) for operation. Alternatively, the main magnet 300 may include a permanent magnet. The main magnet 300 may include a bore 301 that the object is placed within. The main magnet 300 may also control the homogeneity of the generated main magnetic field. Some shim coils may be in the main magnet 300. The shim coils placed in the gap of the main magnet 300 may compensate for the inhomogeneity of the magnetic field of the main magnet 300. The shim coils may be energized by a shim power supply.

The gradient coils 600 may be located inside the main magnet 300. The gradient coils 600 may generate a second magnetic field (or referred to as a gradient field, including gradient fields Gx, Gy, and Gz). The second magnetic field may be superimposed on the main field generated by the main magnet 300 and distort the main field so that the magnetic orientations of the protons of an object varies as a function of their positions inside the gradient field, thereby encoding spatial information into MR signals generated by the region of the object being imaged. The gradient coils 600 may include X coils (e.g., configured to generate the gradient field Gx corresponding to the X direction), Y coils (e.g., configured to generate the gradient field Gy corresponding to the Y direction), and/or Z coils (e.g., configured to generate the gradient field Gz corresponding to the Z direction) (not shown). The three sets of coils may generate three different magnetic fields that are used for position encoding. The gradient coils 600 may allow spatial encoding of MR signals for image construction. All three sets of coils of the gradient coils 600 may be energized and three gradient fields may be generated thereby.

The power supply component 200 of the gradient coil 600 refers to a structure that transmits power to the gradient coil. The power supply component 200 of the gradient coil 600 may be configured to transmit power to the gradient coil 600 together with the cable 100. The power supply component 200 of the gradient coil 600 may be connected with the cable 100 and the gradient coil 600, respectively.

In some embodiments, the power supply component 200 of the gradient coil 600 may include a first insulation device 400. The first insulation device 400 may be equipped with at least one a first conductive unit, which may be connected with the cable 100 and the gradient coil 600, respectively. In some embodiments, the power supply component 200 of the gradient coil may further include a second insulation device 500. The second insulation device 500 may be arranged with a second conductive unit, and the second conductive unit in the second insulation device 500 may be connected with the first conductive unit in the first insulation device 400 and the cable 100, respectively. The current may flow through the cable 100, the second conductive unit, and the first conductive unit in sequence to flow to the gradient coil 600, thereby transmitting power to the gradient coil 600.

The first insulation device 400 refers to a device for connecting the gradient coil 600 and the cable 100. In some embodiments, the first insulation device 400 may be matched with the gradient coil 600 and configured to transmit power to the gradient coil 600.

In some embodiments, the first insulation device 400 may be arranged with a first conductive unit.

The first conductive unit may be a rigid conductor for transmitting power (e.g., current). The rigid conductor refers to a conductor material with a high mechanical property such as strength and hardness, such as copper, steel, copper nickel alloys, and other metal conductors. In some embodiments, the first conductive unit may be in various shapes, such as cylinder, cube, rectangle, sheet, or the like.

It should be understood that if the first conductive unit is a flexible conductor (e.g., a cable, etc.), a connection between the gradient coil 600 and the first conductive unit is a soft connection. The connection stability of the soft connection is relatively poor. When the first conductive unit is located in a magnetic field and subjected to Lorentz force, the flexible first conductive unit is prone to fracture, which affects a normal operation of the magnetic resonance imaging system. By arranging the first conductive unit as a rigid conductor, a hard connection between the first conductive unit and the gradient coil 600 is achieved, which can ensure the connection stability between the first conductive unit and the gradient coil 600.

In some embodiments, the first conductive unit may include a first end and a second end. The first end of the first conductive unit may be electrically connected with the gradient coil 600 of the magnetic resonance imaging system, and the second end of the first conductive unit may be electrically connected with the cable 100 of the magnetic resonance imaging system. The electrical connection refers to a connection that enables the flow of energy (e.g., current) between components. The electrical connection of components may be a direct connection, e.g., there are no intermediate components between the components; the electrical connection of components may also be an indirect connection, e.g., there is at least one intermediate component between the components.

In some embodiments, an arrangement of the first conductive unit may correspond to a preset voltage phase distribution. The voltage phase distribution may be a sequence in which the voltage phases are arranged, for example, the voltage phase distribution may be (V₁⁺, V₁⁻), or the like. In some embodiments, the voltage phase distribution may be preset based on experience or demand. In some embodiments, for the cable configured to transmit power to the gradient coil 600, the voltage phase distribution of the cable may be preset. The arrangement of the first conductive unit may be determined based on a preset voltage phase distribution of the cable, and the first insulation device 400 may be configured based on the arrangement of the first conductive unit, for example, the arranged first conductive unit may be poured to obtain the first insulation device 400. For example, N sets of cables may be set to transmit power to the gradient coil 600, and the voltage phase distribution of the N sets of cables may be arranged as (V₁⁺, V₁⁻, . . . , V_{N}⁺, V_{N}) in sequence. Accordingly, 2N first conductive units corresponding to the cables may be arranged in the first insulation device 400, and the voltage phase distribution of the 2N first conductive units may correspond to V₁⁺, V₁⁻, . . . , V_{N}⁺, V_{N}⁻, respectively.

By keeping the arrangement of the first conductive units in the first insulation device 400 consistent with the preset voltage phase distribution, each first conductive unit in the first insulation device 400 is matched with the connected cable, which can facilitate the power supply for the gradient coils 600 in different directions and ensure the normal operation of the magnetic resonance imaging system.

In some embodiments, the first insulation device 400 may further include an insulated fixing module. The insulated fixing module refers to a component used to fix the first conductive unit. In some embodiments, the insulated fixing module may be made of various materials, such as epoxy resin, polyester resin, polycarbonate, and other insulated and hard materials.

In some embodiments, the insulated fixing module may be arranged to wrap the first conductive unit. In some embodiments, the insulated fixing module may be a hollow structure. The external shape of the insulated fixing module may be of various shapes, such as a rectangular structure, an arc structure, an arc trapezoidal structure, or the like. The hollow structure may be configured to accommodate the first conductive unit, and the shape of the hollow structure (the internal shape of the insulated fixing module) may be matched with the shape of the first conductive unit. In some embodiments, the external shape of the insulated fixing module may be matched with the shape of the gradient coil 600. For example, a radian of the external shape of the insulated fixing module may be the same as a radian of the shape of the gradient coil 600.

By arranging the insulated fixing module, the first conductive unit is effectively fixed, an insulation effect of the first insulation device 400 can be provided, and the stability and safety of the first insulation device 400 can be improved.

In some embodiments, the insulated fixing module and the first conductive unit may be integrally formed. Exemplary integrated forming process may include injection molding, pouring, 3D printing, or the like. Merely for example, a plurality of first conductive units may be arranged in a first insulation device mold according to a corresponding arrangement sequence, and molten epoxy resin may be poured into the first insulation device mold. After the epoxy resin solidifies, the insulated fixing module and the first conductive unit that are integrally formed may be obtained.

By integrally forming the insulated fixing module and the first conductive unit, the stability of the first conductive units can be ensured and problems such as displacement between the first conductive units and fracture of the first conductive units can be avoided.

In some embodiments, the first insulation device 400 may be further arranged with a cooling pipe. The cooling pipe may be a channel type (e.g. tubular) device configured to accommodate the cooling medium. The cooling medium may be liquid (e.g., water, ethanol, etc.), gas (e.g., nitrogen, etc.), or the like.

In some embodiments, the cooling pipe may be in various shapes, such as a straight tube shape, a curved tube shape, or the like. In some embodiments, a shape of the cooling pipe may be matched with an extended shape of the first conductive unit, so that the first insulation device 400 and gradient coil 600 may be well fixed when fixing the first insulation device 400 and the gradient coil 600 subsequently, for example, when the extension shape of the first conductive unit is in a shape of an arc ladder, the cooling pipe may be also in a corresponding arc ladder shape.

In some embodiments, the cooling pipe may be configured to cool the first conductive unit. In some embodiments, the cooling pipe may be fixed on the first conductive unit; or, the cooling pipe may be arranged around the first conductive unit.

In some embodiments, the insulated fixed module may wrap around the cooling tube.

Merely for example, when the insulated fixing module and the first conductive unit are integrally formed, the cooling pipe may be arranged in the first insulation device mold, and the insulated fixing module, the cooling pipe, and the st conductive unit may be integrally formed. In some embodiments, the cooling pipe may be connected to a cooling pipe in the gradient coil of the main magnet to form a cooling circulation system.

By arranging the cooling pipe, the cooling medium is introduced into the cooling pipe to avoid adverse effects such as insulated fixing module melting caused by high temperature of the first conductive unit and extend a service life of the first insulation device. By arranging the insulated fixing module to wrap around the cooling pipe, a misaligned movement of the cooling pipe can be prevented.

It should be understood that the main magnet 300 may include a circular aperture (i.e. a scanning bore 301), and the gradient coil 600 may be arranged within the circular aperture of the main magnet 300, so the shape of the gradient coil 600 may have radian.

In some embodiments, the first insulation device 400 may include an arc-shaped structure. The radian of the arc-shaped structure may be matched with the radian of the gradient coil 600. Specifically, a spiral coil made of wires may be poured to form a gradient coil 600, which may be in the shape of a tube. A radian of a surface of the gradient coil 600 may be measured and a forming cavity is set based on the measured radian. The forming cavity may be an arc (e.g., a semi circular or a circular), and the radian of the forming cavity may be matched with the radian of the gradient coil 600 (e.g., with a same radian as the gradient coil 600). The first conductive unit may be fixed in the forming cavity, and the forming cavity may be poured to form an arc-shaped structure, and the arc-shaped structure (i.e., the insulated fixing module) and the first conductive unit may form the first insulation device.

By making that the first insulation device includes an arc-shape structure, a radian of the arc-shape is matched with a radian of the gradient coil 600, so that the first insulation device may be adapted to the gradient coil, which can facilitate an establishment of the electrical connection between the first insulation device and the gradient coil 600, and facilitate the power supply component 200 of the gradient coil to supply power to the gradient coil.

It should be noted that the first insulation device 400 is not limited to including an arc-shaped structure, but can also include other geometric structures, as long as it can be electrically connected as the gradient coil 600. For example, the first insulation device 400 may also include a rectangular or cubic body, or the like. Taking a rectangular body as an example, when a tube wall of the tubular gradient coil is relatively thick, the forming cavity may be arranged as a rectangular body, which may be poured to obtain a rectangular structure. The first insulation device 400 may include the rectangular structure and the first conductive unit.

In some embodiments, the first insulation device 400 may be fixedly connected with the gradient coil 600. The fixed installation process may include but is not limited to, nailing, riveting, welding, or integrated forming.

In some embodiments, the first insulation device 400 and the gradient coil 600 may be integrally formed. The specific integrally formed process may include injection molding, pouring, 3D printing, or the like.

In some embodiments, a process of integrally forming the first insulation device 400 and the gradient coil 600 may include: integrally forming the insulated fixing module and the first conductive unit to obtain the first insulation device 400; connecting the first insulation device 400 and the gradient coil 600 fixedly; integrally forming the first insulation device 400 and the gradient coil 600 that are fixedly connected using an integrated forming device.

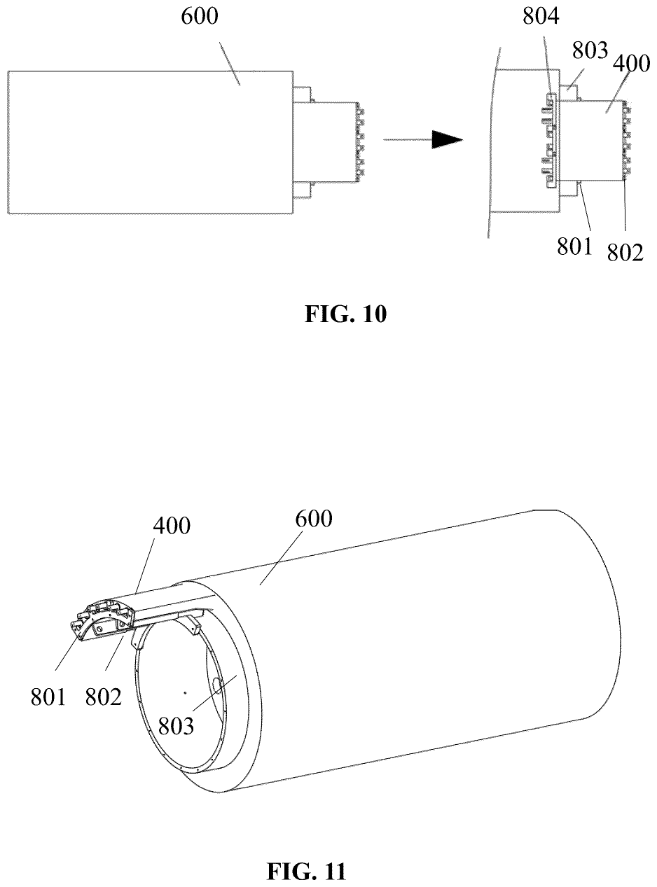

More descriptions of the process of integrally forming the insulated fixing module and the first conductive unit to obtain the first insulation device 400 may be found elsewhere in the present disclosure (e.g., in connection with the description of FIGS. 10-12), which may not be repeated herein.

In some embodiments, the first insulation device 400 may be fixedly connected with the gradient coil 600 in various ways. For example, the first insulation device 400 may be fixed into the gradient coil forming mold 803 to achieve a fixed connection between the first insulation device 400 and the gradient coil 600. As another example, the fixed connection between the first insulation device 400 and the gradient coil 600 may be achieved through bonding, nailing, and other manners.

In some embodiments, the power supply component 200 of the gradient coil may also include a connecting component.

The connecting component refers to a component that fixedly connects the first insulation device 400 and the gradient coil 600. In some embodiments, the connecting component may be made of non-metallic materials with higher strength, such as epoxy resin, or the like. In some embodiments, the connecting component may be connected with a first end of the first conductive unit to prevent the first end of the first conductive unit from being separated from the gradient coil 600. The specific connection manners may include but are not limited to nailing, riveting, or the like.

In some embodiments, as shown in FIG. 10, the connecting component 804 may be connected with the first insulation device 400 through screws for fixing the first insulation device 400 to the gradient coil 600.

By arranging the connection component, a fixed connection between the first insulation device and the gradient coil 600 can be achieved, the first end of the first conductive unit from being separated from the gradient coil 600 can be prevented to affect the effect of the integrally formed process.

In some embodiments, an integrally formed process of the first insulation device 400 and gradient coil 600 that are fixedly connected may include but not limited to injection molding, pouring, 3D printing, or the like. For example, the first insulation device 400 and the gradient coil 600 that are fixedly connected may be integrally formed using an integrated forming device.

In some embodiments, as shown in FIGS. 10-12, the integrated forming device may include a gradient coil forming mold 803, a first supporting component 802, and a second supporting component 801.

The gradient coil forming mold 803 may be configured to accommodate the gradient coil 600 and a portion of the first insulation device 400 connecting with the gradient coil 600. It should be understood that the gradient coil 600 and the first insulation device 400 are connected through an integral pouring molding process, when the gradient coil 600 is poured, there is a position for placing the insulation device 400 is retained on the gradient coil forming mold 803, which can support the first insulation device 400 and the gradient coil 600 simultaneously for pouring together.

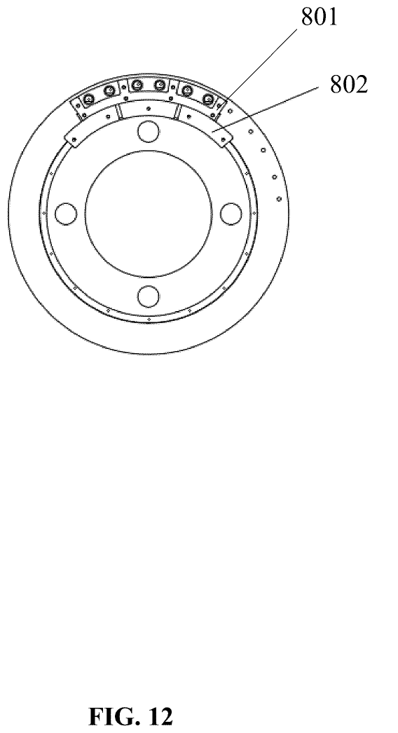

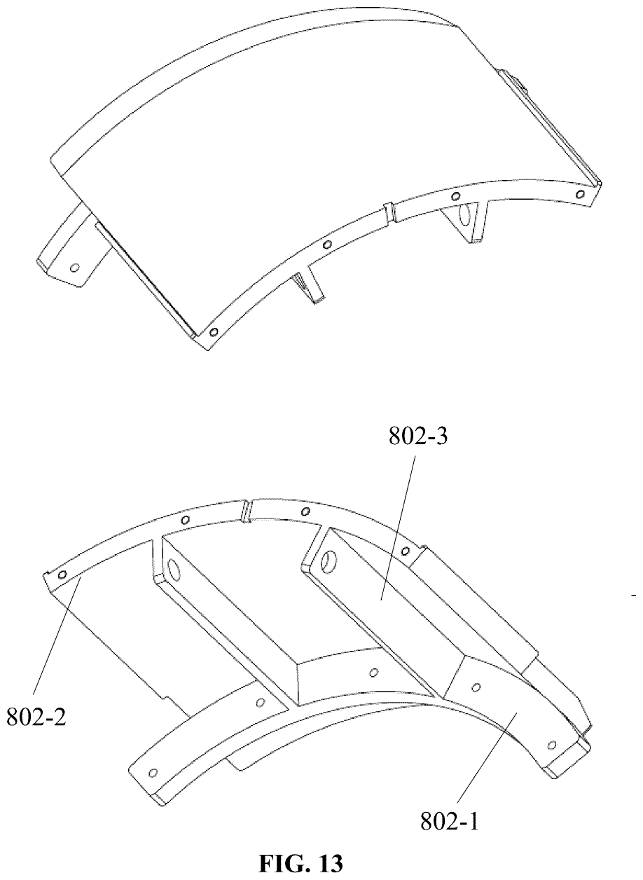

The first supporting component 802 may be configured to support the first insulation device 400, and the first supporting component 802 may be connected with the gradient coil forming mold 803. In some embodiments, the first supporting component 802 may be made of various materials, such as aluminum alloy, or the like. The first supporting component 802 may be in various shapes, for example, as shown in FIGS. 12 to 13, the first supporting component 802 may be an arc-shaped groove structure matched with the gradient coil 600. In some embodiments, a thickness of the first supporting component 802 may be matched with the gradient coil forming mold 803. For example, the thickness of the first supporting component 802 may be the same as a thickness of the gradient coil forming mold 803.

The second supporting component 801 may be configured to support the first insulation device 400, and the second supporting component 801 may be connected with the first supporting component 802 and the first insulation device 400. In some embodiments, the second supporting component 801 may be made of various materials, such as aluminum alloy, or the like. The second supporting component 801 may be in various shapes, for example, the second supporting component 801 may be an arc-shaped structure matched with the first insulation device 400.

In some embodiments, the second supporting component 801 may include at least one groove for accommodating the first conductive unit. For example, as shown in FIG. 12, the second supporting component 801 may include three grooves each of which is configured to accommodate two of six first conductive units.

By arranging at least one groove on the second supporting component, the first conductive unit can be accommodated.

In some embodiments, a first end of the first supporting component 802 may be connected with an end surface of the gradient coil forming mold 803 closed to the first insulation device 400.

The second supporting component 801 may be connected with a second end of the first supporting component 802 and a second end of the first insulation device 400. The first end of the first supporting component 802 is opposite to the second end of the first supporting component 802, and the second end of the first insulation device 400 may correspond to the second end of the first conductive unit.

As shown in FIGS. 10-12, the first supporting component 802 may be arranged on an end surface of the gradient coil forming mold 803 corresponding to the first insulation device 400, and the second supporting component 801 may be installed on the first supporting component 802; the first insulation device 400 may be placed on the first supporting component 802, and the second supporting component 801 may be fixedly connected with the first insulation device 400 in a fixing manner to achieve a relatively good fixation of the first insulation device 400. The specific fixed connection manners may be nailing, riveting, welding, or the like.

Merely for example, the first supporting component 802 may be arranged on the end surface of the gradient coil forming mold 803 corresponding to the first insulation device 400, and the second supporting component 801 may be arranged on the first supporting component 802; the gradient coil 600 may be placed in the gradient coil forming mold 803, the first insulation device 400 may be placed on the first supporting component 802, the gradient coil 600 may be fixedly connected with the first insulation device 200, and the second supporting component 801 may be fixedly connected with the first insulation device 400 for integrated forming.

In some embodiments, the second supporting component 801 and the first supporting component 802 may be integrally formed.

In some embodiments, after the integrally forming is completed, the second supporting component 801 and the first supporting component 802 may be removed from the first insulation device 400.

In some embodiments, as shown in FIG. 13, the first supporting component 802 may include a connection portion 802-1, a support portion 802-2, and a strengthening portion 802-3. The connection portion 802-1 may be configured to connect with the gradient coil forming mold 803. The connection portion 802-1 may be connected with an end surface of the gradient coil forming mold 803 closed to the first insulation device 400. The connection portion 802-1 may be an arc-shaped structure matched with the gradient coil 600. The support portion 802-2 may be configured to support the first insulation device 400 during the process for integrally forming the first insulation device 400 and the gradient coil 600. The support portion 802-2 may be connected with the connection portion 802-1. The support portion 802-2 may be an arc-shaped structure matched with the gradient coil 600. The strengthening portion 802-3 may be configured to strengthen the support effect of the support portion 802-2. The strengthening portion 802-3 may be connected with the connection portion 802-1 and the support portion 802-2.

By arranging that the integrated forming device includes a gradient coil forming mold 803, a first supporting component 802, and a second supporting component 801, a relatively good fixation of the first insulation device 400 and the gradient coil forming mold 803 is achieved, which can facilitate the subsequent execution of the integrally forming process and effectively avoid the detachment of the first insulation device 400 and the gradient coil 600 due to improper fixation, to avoid affecting the integrally forming effect.

In some embodiments, as shown in FIG. 3, the first conductive unit, the insulated fixing module, and the cooling pipe may be integrally formed to obtain the first insulation device 400, the first insulation device 400 may be fixed on the gradient coil forming mold 803 based on the aforementioned manners, and a gradient coil 609 fixedly arranged with the first insulation device 400 may be obtained by pouring the gradient coil forming mold 803, which achieves the integrally forming of the first insulation device 400 and the gradient coil. The specific pouring material may be insulation material, such as epoxy resin, or the like.

By integrally forming the first insulation device and the gradient coil, a misaligned movement between the first insulation device and the gradient coil, which can ensure stability of the electrical connection components between the first insulation device and the gradient coil, and ensure the normal operation of the power supply.

In some embodiments, the first insulation device 400 may be located within the main magnet 300 of the magnetic resonance imaging system. For example, the first insulation device 400 may be placed within a circular aperture of the main magnet 300.

In some embodiments, a length of the first insulation device 400 along an axial direction of the magnetic resonance imaging system may be smaller than a target distance.

In some embodiments, along the axial direction of the magnetic resonance imaging system, the gradient coil 600 may include a first end, the main magnet 300 of the magnetic resonance imaging system may include a second end, and the first end may correspond to the second end. Merely for example, as shown in FIG. 3, the axial direction of the magnetic resonance imaging system may be parallel to the Z axis, a first end of the gradient coil 600 may be 601, and a second end of the main magnet 300 may be 302. Therefore, the target distance may be a distance along the Z axis between the first end 601 and the second end 302, which is a distance B in FIG. 3.

By arranging a length of the first insulation device along the axial direction of the magnetic resonance imaging system to be less than the target distance, problems such as installation difficulties caused by the length of the first insulation device being too long can be avoided.

In some embodiments, a ratio of the length to the target distance may be 0.5-0.8.

By arranging the ratio of the length of the first insulation device to the target distance to be 0.5-0.8, it is convenient to set a reasonable installation position of the first insulation device, a length of the first conductive unit, or the like, which can minimize the influence of the magnetic field on the first insulation device.

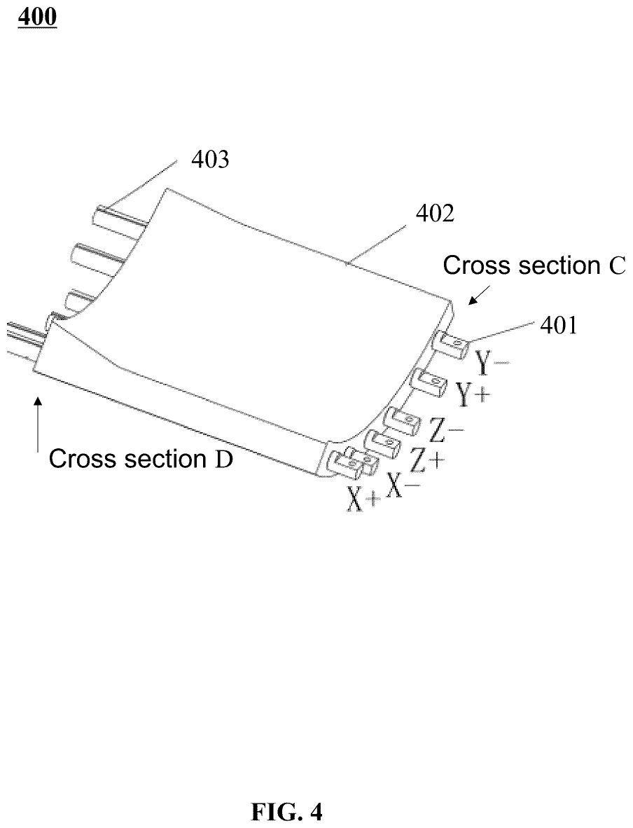

In some embodiments, along the axial direction of the magnetic resonance imaging system, the first insulation device 400 may include a first end and a second end; the first end of the first insulation device 400 may correspond to the first end of the first conductive unit, and the second end of the first insulation device 400 may correspond to the second end of the first conductive unit; a cross section of the first end of the first insulation device 400 may be smaller than a cross section of the second end of the first insulation device 400. In some embodiments, as shown in FIG. 4, the cross section of the first end of the first insulation device 400 may be a section C, the cross section of the second end of the first insulation device 400 may be a section D, and the section C may be smaller than the section D.

By setting the cross section of the first end of the first insulation device to be smaller than the cross section of the second end of the first insulation device, it is possible to achieve a relatively good axial fixation of the first insulation device along the magnetic resonance imaging system.

By setting the power supply component of the gradient coil including the first insulation device, a connection between the power supply component of the gradient coil and the gradient coil has a relatively good stability, preventing the detachment of the cable and gradient coil caused by the magnetic field, which affects the normal power supply of the gradient coil.

In some embodiments, the power supply component 200 of the gradient coil may also include a second insulation device 500.

The second insulation device 500 refers to a device for connecting the cable and the first insulation device 400. In some embodiments, the second insulation device 500 may be configured to achieve a turning and connection of the cable at the circular aperture of the main magnet 300.

In some embodiments, the second insulation device 500 may be arranged with at least one second conductive unit.

The second conductive unit may be a conductive component that transmits current, such as an electrode, or the like. In some embodiments, the second conductive unit may be a good conductor of softer materials, such as soft copper alloys, aluminum, tin, and other metal materials. It should be understood that material used for setting the second conductive unit is relatively soft, which can enable the second conductive unit to withstand a certain amplitude of strength without fatigue fracture.

In some embodiments, the second conductive unit may have various shapes, such as sheet, strip, rectangular, or the like. When the shape of the second conductive unit is sheet, a thickness of the second conductive unit may be 5 mm.

In some embodiments, the first end of the second conductive unit may be electrically connected with the cable of the magnetic resonance imaging system, and the second end of the second conductive unit may be electrically connected with the second end of the first conductive unit.

In some embodiments, the arrangement of the second conductive unit in the second insulation device 500 may correspond to the voltage phase distribution. More descriptions of making the arrangement of the second conductive unit in the second insulation device 500 corresponding to the voltage phase distribution may be found elsewhere, which may not be repeated herein.

In some embodiments, the second conductive unit may include a first portion and a second portion. In some embodiments, materials of the first portion and the second portion may be flexible materials (e.g., polyvinyl chloride, polyethylene, etc.) and may be arranged in a curved shape to absorb vibrations. The first portion refers to a portion where the second end of the second conductive unit is located, which may extend into the main magnet 300 and be connected with the first insulation device 400. In some embodiments, the connection manners between the first portion and the first insulation device 400 may include but are not limited to screw connection, riveting, welding, or the like. The first portion may be configured to absorb vibration from the first insulation device to avoid or reduce relative movement between the first insulation units and/or relative movement between the first insulation unit and the insulated fixing module.

In some embodiments, the length of the first portion may be 100-150 mm to achieve a relatively good absorption of vibration from the first insulation device. The length of the first portion refers to a length of the first portion along the Z axis direction. It should be understood that if the length of the first portion is too short, the ability to absorb vibration may be relatively limited, if the length of the first portion is too long, it is not suitable for bending operation, and the vibration amplitude of the first portion may be caused to be too large, resulting in poor absorption of vibration. Therefore, the length of the first portion needs to be within an appropriate range to ensure the absorption of vibration.

The second portion refers to a portion wherein the first end of the second conductive unit is located, which may be fixed on the end surface of the main magnet.

In some embodiments, there may be a preset angle between the first portion and the second portion. The preset angle may be no less than 70 degrees, such as 80 degrees, 90 degrees, 105 degrees, or the like.

It should be understood that if the preset angle is too small, interference between the second conductive unit and the main magnet 300 can be caused at the circular aperture, making them unable to fit well, affecting the experience of the user and the lifespan of the second conductive unit.

In some embodiments, the length of the second portion may be matched with the radian of the gradient coil 600. The length of the second portion refers to a length of the second portion along the Y axis direction.

It should be understood that the second conductive unit is bent, while the second portion is fixed on the end surface of the main magnet. Due to the curvature of the gradient coil 600, in order to match the second portion fixed on the end surface of the main magnet with the gradient coil 600, the length of the second portion of multiple second conductive units may be different, and the specific length may be set according to the radian of the gradient coil 600.

In some embodiments, a width of the second conductive unit (503, 504, 505, 506, 507, 508) may be negatively correlated with a length of the second portion. The width of the second conductive unit refers to a width of the second conductive unit along the X axis direction. For example, if the second portion of the second conductive unit 508 is longer than the second portion of the second conductive unit 506, the second conductive unit 506 may be set to be wider than the second conductive unit 508.

It should be understood that in order to match the radian of the gradient coil 600, the length of the second portion of multiple second conductive units may be different, the shorter the length of the second portion, the higher the natural frequency of vibration of the second portion and the easier it is to break. By setting the width of the second conductive unit (503, 504, 505, 506, 507, 508) to be negatively correlated with the length of the second portion, a second conductive unit with a shorter second portion can have a wider width, the stiffness of multiple second conductive units is roughly the same, thereby ensuring the absorption of vibration by the second conductive unit and avoiding an increase in vibration amplitude caused by insufficient stiffness of the second conductive unit, which affects the service life of the second conductive unit.

In some embodiments, the second insulation device 500 may be fixed on an end surface of the main magnet of the magnetic resonance imaging system. In some embodiments, the end surface of the main magnet refers to a cross section corresponding to the second end of the main magnet 300, such as a cross section corresponding to the second end 302 of the main magnet 300 in FIG. 3.

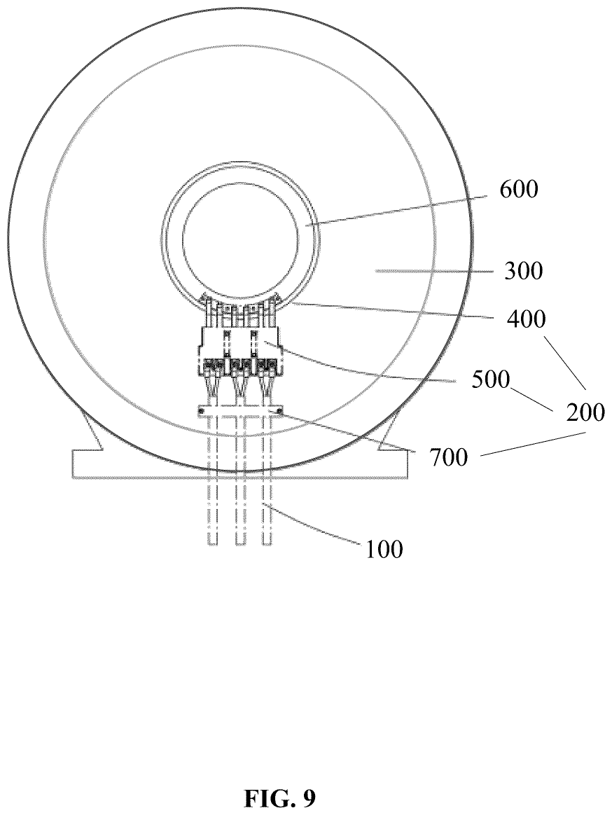

In some embodiments, the first insulation device 400 and the second insulation device 500 may be disposed on the same side of the gradient coil 600 of the magnetic resonance imaging system. In some embodiments, the power supply assembly 200 of the gradient coil 600 may be disposed in any radial direction of the gradient coil 600 of the magnetic resonance imaging system. For example, as shown in FIG. 1, the power supply assembly 200 is disposed in 12 o'clock direction (e.g., the positive Y direction in FIG. 1) of the gradient coil 600. As another example, as shown in FIG. 9, the power supply assembly 200 is disposed in 6 o'clock direction (e.g., the negative Y direction in FIG. 1) of the gradient coil 600. The power supply assembly 200 may also be disposed in 3 o'clock direction (e.g., the negative X direction in FIG. 1) of the gradient coil 600, 9 o'clock direction (e.g., the positive X direction in FIG. 1) of the gradient coil 600, 2 o'clock direction of the gradient coil 600, 5 o'clock direction of the gradient coil 600, 8 o'clock direction of the gradient coil 600, 11 o'clock direction of the gradient coil 600, etc.

Traditional cables may not achieve good turning points at the circular aperture of the main magnet due to space constraints or excessive bending angles. By setting the power supply component of the gradient coil including the second insulation device 500, it is more convenient to achieve a preset angle turning at the circular aperture of the main magnet, which can effectively absorb the vibration effect caused by the magnetic field to avoid relative movement between the first insulation units, and make an electrical connection between the first insulation device and the cable more stable.

In some embodiments, the power supply component 200 of the gradient coil may include a fixing component for achieving fixation between the power supply component 200 of the gradient coil and the main magnet 300.

In some embodiments, the power supply component 200 of the gradient coil may include a first fixing component. The first fixing component may be arranged on the end surface of the main magnet to fix the second insulation device 500 on the end surface of the main magnet. The first fixing member may include but is not limited to bolt, nut, pin, hook, clamp, self-locking pressure plate, hoop, strap, movable ring, or fixing ring. Specifically, a first fixing component may be installed on the end surface of the second insulation device 500 and the main magnet of the magnetic resonance imaging system, or only on the second insulation device 500, or only on the end surface of the main magnet.

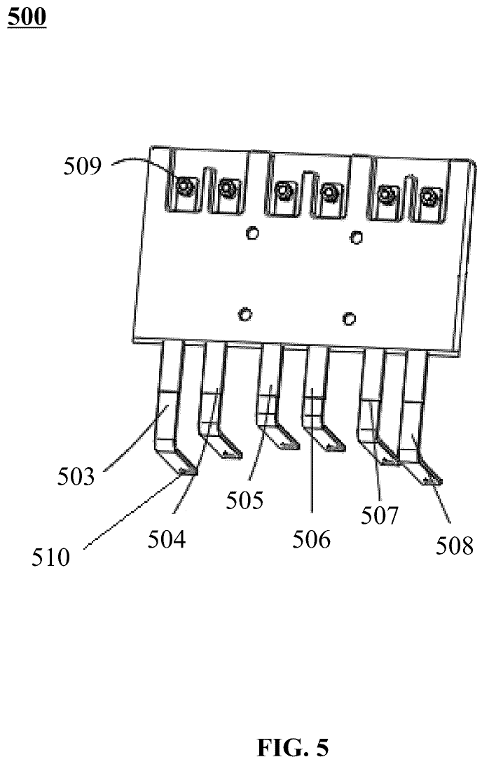

In some embodiments, as shown in FIGS. 2 and 5, screw holes may be set on the second insulation device 500, and a protruding cylinder may be welded on an outer wall of the main magnet end face. A center of the cylinder may be a threaded hole. By passing a screw through the screw hole on the second insulation device 500 and the threaded hole on the main magnet 300, the second insulation device 500 may be fixed on the end surface of the main magnet. The count of screw holes and cylinders may both be 4.

In some embodiments, the end surface of the main magnet may be arranged with a clamp, a size of the clamp may be matched with the second insulation device 500, and the second insulation device 500 may be fixed on the end surface of the main magnet in a clamping manner.

In some embodiments, the second insulation device 500 may be further arranged with a strap, and the strap may be configured to fix the second insulation device 500 on the end surface of the magnet 300.

By setting a first fixing member on the end surface of the main magnet of the second insulation device and/or magnetic resonance imaging system, the second insulation device is fixed on the end surface of the magnet through the first fixing component, thereby fixing the power supply component of the gradient coil and solving the problem of cables being prone to breakage in the magnetic resonance imaging system.

In some embodiments, as shown in FIGS. 1-3 and 6, the power supply component 200 of the gradient coil may also include a second fixing member 700 for fixing the cable 100 to the end surface of the main magnet.

FIG. 7 is a schematic diagram illustrating a structure of a second fixing component according to some embodiments of the present disclosure. As shown in FIG. 7, the second fixing component 700 may include a first crimping plate 701 and a second crimping plate 702, a size of the first crimping plate 701 may be matched with a size of the second crimp plate 702 and the first crimping plate 701 and the second crimping plate 702 may be reserved with holes for the cable 100 to pass through. For example, three holes may be reserved between the first crimping plate 701 and the second crimping plate 702 for three sets of cables to pass through. In some embodiments, additional holes may be provided on the second fixing component 700 configured to fix the second fixing component 700 on the end surface of the main magnet. For example, the second fixing component 700 may be arranged with two threaded holes to fix the second fixing component 700 on the end surface of the main magnet through a screw connection.

By setting that the power supply component of the gradient coil further includes the second fixing component, the second fixing component fixes each set of cables on the end surface of the main magnet, which can solve the problem of the cable being prone to breakage in the magnetic resonance imaging system.

FIG. 4 is a schematic diagram illustrating a structure of a first insulation device according to some embodiments of the present disclosure.

As shown in FIG. 4, an insulation material (e.g., epoxy resin) may be configured to integrally form (e.g., pour) and fix the first conductive unit 401 to form an insulated fixing module 402 that wraps around the first conductive unit, thereby obtaining the first insulation device 400. Two end of the first insulation unit 401 may extend from the first insulation device 400 and connect the first insulation device 400 with the cable 100 and the gradient coil 600, respectively. Specifically, the cable 100 may be divided into three sets, which may correspond to terminals X+ and X− of the X coils of the gradient coil 600, terminals Y+ and Y− of the Y coils of the gradient coil 600, and terminals Z+ and Z− of the Z coils of the gradient coil 600. Six first conductive units 401 may be set, and a voltage phase distribution from left to right may be X+, X−, Z+, Z−, Y+, Y−. The six first conductive units 401 may be fixed in the forming cavity and poured with insulation materials (e.g., epoxy resin) to solidify and form. When the gradient coil 600 is a tubular, to be matched with the gradient coil 600, the first insulation device 400 may be poured into an arc-shaped structure. Before pouring, the cooling pipe 403 may also be arranged (e.g., fixed on or around the first conductive unit 401) to cool the first conductive unit 400 through the cooling pipe 403. In order to ensure a good connection between the first conductive unit 400 and the gradient coil 600, as shown in FIG. 4, the shape of the first end of the first insulation unit 401 may be designed as an arc-ladder shape, and the cross section C of the first end of the first insulation device 400 may be smaller than the cross section D of the second end of the first insulation device 400, which can achieve a good fixation of the first conductive unit 400 and the axial direction of the gradient coil.

FIG. 5 is a schematic diagram illustrating a structure of a second insulation device according to some embodiments of the present disclosure.

As shown in FIG. 5, the six second conductive units 503-508 may be arranged corresponding to the voltage phase distribution of the first conductive unit 401, wherein 503 represents X+, 504 represents X−, 505 represents Z+, 506 represents Z−, 507 represents Y+, and 508 represents Y−. The six second conductive units may also be bent, specifically, the six second conductive units may be bent into 90°. The second portion may be poured into the forming cavity to obtain a second insulation device 500, which may extend into a circular aperture of the main magnet 300 and may be electrically connected with the first insulation device 400. The length of the first portion may be calculated according to actual needs, for example, the length of the first portion may be controlled within a range of 100-150 mm to absorb the vibration.

In some embodiments, the second conductive unit may be arranged with a first end and a second end, and the cable may be arranged with a third end. The first end and second end of the second conductive unit may be connected with the third end of the cable and the second end of the first conductive unit in the first insulation device 400, respectively. The specific connection manner may include but is not limited to a threaded connection. As shown in FIG. 5, the first end of the second conductive unit and the second end of the second conductive unit may include but are not limited to a hole position 509 (i.e., a first end) and a hole position 510 (i.e., a second end) may be arranged at both ends of each second conductive unit.

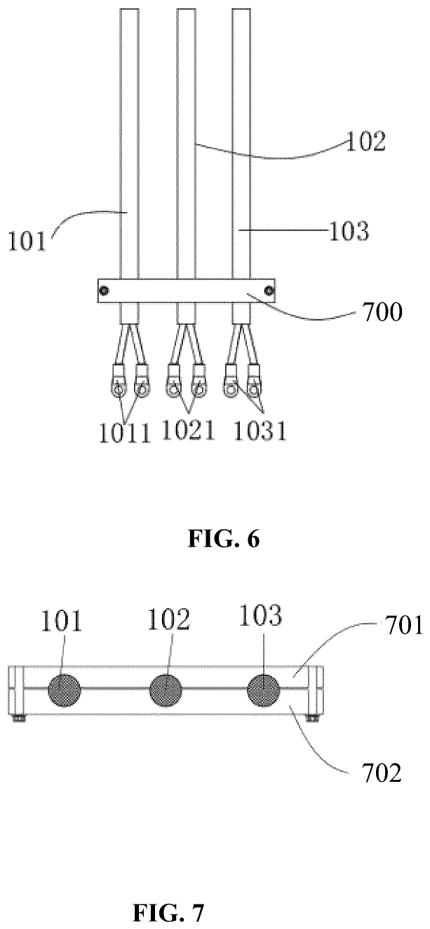

FIG. 6 is a schematic diagram illustrating a structure of a cable according to some embodiments of the present disclosure. As shown in FIG. 6, the third end of the cable may be but not limited to a wiring terminal or a wiring terminal crimped at a bottom of the cable (i.e., the third end), for example, a wiring terminal 1011 may be crimped at the bottom of cable 101, a wiring terminal 1021 may be crimped at the bottom of cable 102, and a wiring terminal 1031 may be crimped at the bottom of cable 103. The six wiring terminals may correspond to the voltage phase distribution X+, X−, Z+, Z−, Y+, and Y−, respectively. In some embodiments, the hole position at the first end of the second conductive unit may be crimped together with the wiring terminal of the cable through a locking nut to achieve a connection between the cable and the second conductive unit, and a connection between the second conductive unit and the first conductive unit may be achieved by connecting the hole position of the second end of the second conductive unit with the second end of the first conductive unit through the locking nut.

By setting a first end and a second end on the second conductive unit, and a third end on the cable, the first end and the second end of the second conductive unit may be connected with the third end of the cable and the second end of the first conductive unit, respectively, a connection between the cable and the second conductive unit and a connection between the second conductive unit and the first conductive unit can be achieved, which can facilitate the power supply component of the gradient coil to supply power to the gradient coil.

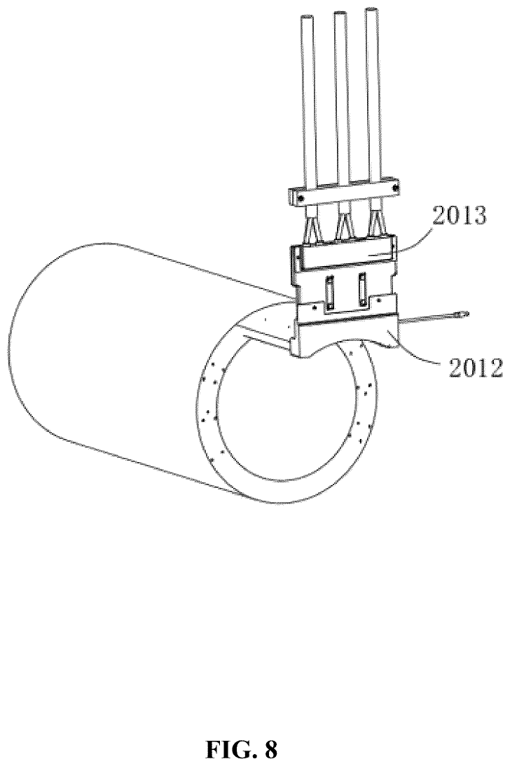

In some embodiments, the second insulation device 500 may be arranged with a safety shield. FIG. 8 is a schematic diagram illustrating a structure of a safety shield of a second insulation device according to some embodiments of the present disclosure. As shown in FIG. 8, an upper portion of the second insulation device 500 may be arranged with a first safety shield 2012, and a lower portion of the second insulation device 500 may be arranged with a second safety shield 2013. Both the first safety shield 2012 and the second safety shield 2013 may be made of insulation material.

By setting the safety shield on the second insulation device, the safety of the power supply component of the gradient coil can be improved and the risk of electric shock can be reduced.

In some embodiments, the power supply component 200 of the gradient coil may be oriented in any direction toward the end surface of the main magnet. Specifically, the power supply component 200 of the gradient coil may face toward a ceiling or ground direction on the end surface of the main magnet, and may also face towards a horizontal or other direction on the end surface of the main magnet.

As shown in FIG. 1, when the power supply component 200 of the gradient coil is arranged towards the ceiling direction on the end surface of the main magnet, the cable may be arranged at the ceiling.

FIG. 9 is a schematic diagram illustrating a structure of a power supply assembly of a gradient coil facing the ground according to other embodiments of the present disclosure.

As shown in FIG. 9, the power supply component 200 of the gradient coil may be arranged towards the ground on the end surface of the main magnet, and the cable may be arranged on the ground.

By orienting the power supply component of the gradient coil towards the end surface of the magnet in any direction, the cable may be flexibly arranged, which can improve the flexibility of using the power supply component of the gradient coil.

In some embodiments, the first insulation device may include a conductive pillar insulation device matched with the gradient coil of the magnetic resonance imaging system. The second insulation device may include an electrode insulation device.

The electrode insulation device may be arranged with an electrode, and the conductive pillar insulation device may be arranged with a conductive pillar; the electrode may be connected with the cable 100 of the magnetic resonance imaging system and the conductive pillar in the conductive pillar insulation device to supply power to the gradient coil 600.

The cable 100 may be a current transmission device, such as a wire.

The electrode insulation device may be a device with an internal electrode and an external insulation material.

The conductive pillar insulation device may be a device with an internal conductive pillar and an external insulation material.

The gradient coil 600 may be a coil capable of generating a gradient magnetic field.

The electrode may be a device configured to transmit current, specifically, the electrode may be a good conductor and made of a softer material.

The conductive pillar may be a conductor used to transmit current.

FIG. 2 is a schematic diagram illustrating a front view of a magnetic resonance imaging system according to some embodiments of the present disclosure; FIG. 3 is a schematic diagram illustrating a cross section view of a magnetic resonance imaging system according to some embodiments of the present disclosure. According to FIGS. 1 to 3, the power supply component of the gradient coil may be applied to the magnetic resonance imaging system, which may include a cable 100, a main magnet 300, a gradient coil 600, and a power supply component of the gradient coil. The main magnet 300 may be configured to generate a magnetic field, and the gradient coil 600 may be arranged inside the main magnet 300. The power supply component of the gradient coil may be connected with the cable 100 and the gradient coil 600, respectively. The cable 100 and the gradient supply component of the coil power may be configured to supply power to the gradient coil 600.

The power supply component of the gradient coil may include an electrode insulation device and a conductive pillar insulation device. The electrode insulation device may be arranged with an electrode, and the conductive pillar insulation device may be arranged with a conductive pillar. The shape of the conductive pillar insulation device may be matched with the shape of the gradient coil 600 of the magnetic resonance imaging system, the electrode in the electrode insulation device may be connected with the cable 100 and the conductive pillar in the conductive pillar insulation device, respectively. The current may sequentially flow through the cable 100, the electrode in the electrode strip insulation device, and the conductive pillar in the conductive pillar insulation device to flow to the gradient coil 600, to supply power to the gradient coil 600.

The power supply component of the gradient coil may supply power to the gradient coil 600 by setting the electrode in the electrode insulation device and the conductive pillar in the conductive pillar insulation device, connecting the electrode to the cable 100 and the conductive pillar, respectively, which enables the current to pass through the cable 100, the electrode in the electrode insulation device, and the conductive pillar in the conductive pillar insulation device in sequence to supply power to the gradient coil 600, due to the electrical connection component of gradient coil 600 being fixed through the electrode insulation device and conductive insulation device, the problem of unstable or even easily broken cable in the magnetic resonance imaging system can be avoided.

The main magnet 300 may include a circular aperture, and the gradient coil 600 may be arranged within the circular aperture of the main magnet 300, so the shape of the gradient coil may have an arc. In one embodiment, the conductive pillar insulation device may include an arc-shaped structure, and a radian of the arc-shaped structure may be matched with a radian of the gradient coil. Specifically, a spiral coil made of wires may be poured to form a gradient coil, which may be but is not limited to a tubular. A radian of a surface of the gradient coil may be measured, and a forming cavity may be arranged based on the measured radian, the forming cavity may be curved, such as a half circle or arc-shaped, and the radian of the forming cavity may be matched with the radian of the gradient coil, such as with the same radian as the gradient coil, the conductive pillar may be fixed in the forming cavity, and the forming cavity may be poured to form an arc-shaped structure. The arc-shaped structure and the conductive pillar may form a conductive pillar insulation device. It should be noted that the material used for pouring may be insulation material, such as epoxy resin. In some embodiments, by making the conductive insulation device including an arc-shape structure, the radian of the arc-shaped structure may be matched with the radian of the gradient coil, to match the conductive pillar insulation device with the gradient coil, which can facilitate the establishment of an electrical connection between the conductive insulation device and the gradient coil, and facilitate the power supply component of the gradient coil to supply power to the gradient coil.

It should also be noted that the conductive pillar insulation device is not limited to including an arc-shaped structure, as long as it can be achieved as a gradient coil electrical connection. For example, the conductive pillar insulation device may also include other geometric structures, such as a rectangular or cubic body. Taking a rectangular body as an example, when a pipe wall of the tubular gradient coil is relatively thick, the forming cavity may be arranged as a rectangular body, and the rectangular structure may be poured to form a conductive pillar insulation device. The rectangular structure and the current guide pillar may form the conductive pillar insulation device.

In some embodiments, the conductive pillar insulation device may be fixedly arranged with the gradient coil. The fixed installation manners of the conductive pillar insulation device and the gradient coil may include but are not limited to, nailing, riveting, welding, or pouring.

Specifically, the conductive pillar insulation device and the gradient coil may be poured and formed as a whole. For example, in FIG. 3, the conductive pillar may be poured to form a conductive pillar insulation device, then, the conductive pillar insulation device may be fixed on a pouring mold of the gradient coil, and the pouring mold of the gradient coil may be poured to obtain a gradient coil 600 that is fixed and installed together with the conductive pillar insulation device. It should be noted that the material used for pouring may be insulation material, such as epoxy resin. In some embodiments, by fixedly arranging the conductive pillar insulation device with the gradient coil, a misaligned movement between the conductive pillar insulation device and the gradient coil can be avoided, the stability of the electrical connection component between the conductive pillar and the gradient coil can be ensured, and the reliable power supply of the gradient coil by the power supply component of the gradient coil can be ensured.

In some embodiments, the conductive pillar insulation device may be arranged with a cooling pipe, and the cooling pipe may be configured to cool the conductive pillar. The cooling pipe may be a tubular device used to accommodate cooling substances, such as a cooling water pipe.

Specifically, the conductive pillar insulation device and the gradient coil may be integrally formed, and the cooling pipe may be located around the conductive pillar. Before pouring the conductive pillar insulation device, the cooling pipe may be arranged around the conductive pillar, and the cooling pipe and conductive pillar may be poured together to form a conductive pillar insulation device; the present disclosure does not limit a specific shape of the cooling pile, preferably, the shape of the cooling pipe may be matched with an extended shape of the conductive pillar, to ensure that the conductive pillar insulation device and gradient coil can be well fixed during the subsequent installation of the conductive pillar insulation device and gradient coil, for example, when the extended shape of the conductive pillar is in an arc-ladder shape, the cooling pipe may be in a corresponding arc-ladder shape. In some embodiments, by setting a cooling pipe in the conductive guide pillar insulation device, a cooling substance may be introduced into the cooling pipe to cool the conductive pillar in the conductive pillar insulation device, which can avoid excessive temperature of the conductive pillar.

In some embodiments, the arrangement sequence of the conductive pillar in the conductive pillar insulation device may correspond to the preset voltage phase distribution. The voltage phase distribution may be an arrangement sequence of voltage phases. Specifically, for the cables supplying power to the gradient coil, the voltage phase distribution of the cables may be preset. Based on the preset voltage phase distribution of the cables, the arrangement sequence of the conductive pillars may be determined, and the arranged conductive pillars may be poured to form a conductive pillar insulation device, for example, N sets of cables may be set to supply power to the gradient coil, and the voltage phase distribution of N sets of cables may be set to v1+, v1−, . . . , vN+, and vN−. In the conductive pillar insulation device, 2N conductive pillars corresponding to the cables may be set, and the voltage phase distribution of the 2N conductive pillars may be v1+, v1−, . . . , vN+, and vN−. In some embodiments, by aligning the arrangement sequence of the conductive pillars in the conductive pillar insulation device with the preset voltage phase distribution, each conductive pillar in the conductive pillar insulation device may be adapted to a connected cable, which can facilitate the power supply of gradient coils in different directions and ensure the normal operation of the magnetic resonance imaging system.

FIG. 4 is a schematic diagram illustrating a structure of a first insulation device according to some embodiments of the present disclosure. According to FIG. 4, an insulation material (e.g., epoxy resin) may be configured to pour and form the conductive pillar 401 to obtain a conductive pillar insulation device. The conductive pillar insulation device may lead out a certain length of conductive pillar 401 and connect the conductive pillar 401 with the electrode insulation device to obtain the power supply component of the gradient coil. Specifically, the cables may be divided into three sets, which may correspond three sets of power supply cables X+, X−, Z+, Z−, Y+, Y− of the gradient coil. The six conductive pillars 401 may be set, and the voltage phase distribution from left to right may be X+, X−, Z+, Z−, Y+, Y−. The six conductive pillars 401 may be fixed in the forming cavity and poured with insulation material (e.g., epoxy resin) to solidify and form. When the gradient coil is a tubular, to be matched with the gradient coil, the conductive pillar insulation device may be poured into a semicircle shape. Before the pouring, the cooling pipe 403 may also be arranged around the conductive pillar 401 to cool the conductive pillar insulation device through the cooling pipe 403. In order to ensure a good connection between the conductive pillar insulation device and the gradient coil during subsequent pouring operations, the shape of the cooling pipe 403 may also be designed as an arc-ladder shape, and a volume of a rear of the cooling pipe 403 may be larger than a volume of a front of the cooling pipe 403, which can achieve good fixation of the conductive pillar insulation device and the axial direction of the gradient coil.

In some embodiments, the electrode may be in a bent shape, and the arrangement sequence of the electrode in the electrode insulation device may correspond to the voltage phase distribution. Specifically, the electrode may be bent and arranged according to the preset voltage phase distribution, and one end of the electrode may be poured to form an electrode insulation device. FIG. 5 is a schematic diagram illustrating a structure of a second insulation device according to some embodiments of the present disclosure. According to FIG. 5, six electrodes 503-508 may be arranged corresponding to the voltage phase distribution of the conductive pillar 401, wherein 503 represents X+, 504 represents X−, 505 represents Z+, 506 represents Z−, 507 represents Y+, and 508 represents Y−, the six electrodes may also be bent, specifically, the six electrodes may be bent into a 90° shape, one end may be poured into a forming cavity to form an electrode insulation device, while a length of the bent end (i.e., another end) may be calculated according to actual needs, for example, the length of the bent end may be controlled within a range of 100-150 mm to absorb vibration. In some embodiments, by bending the electrode into a curved shape, the arrangement sequence of the electrodes in the electrode insulation device may correspond to the voltage phase distribution, which can achieve a fixed electrical connection of the power supply component of the gradient coil at a right angle turning of the magnet, which can solve the problem of cables being prone to breakage in the magnetic resonance imaging system.

In some embodiments, the two ends of the electrode may be arranged with a first connecting end and a second connecting end, and the cable may be arranged with a third connecting end. The first connecting end and the second connecting end may be connected with the third connecting end of the cable and the conductive pillar in the conductive pillar insulation device, respectively. A connection between the cable and the electrode may include but is not limited to a threaded connection. A first connecting end and a second connecting end may be arranged at both ends of the electrode, and a third connecting end may be arranged at the cable. The third connecting end of the cable may be connected with the first connecting end of the electrode, and the second connecting end of the electrode may be connected with the conductive pillar. According to FIG. 5, the first connecting end and the second connecting end may be but are not limited to a hole position 509 (i.e., the first connecting end) and a hole position 510 (i.e., the second connecting end) may be arranged at both ends of each electrode. FIG. 6 is a schematic diagram illustrating a structure of a cable according to some embodiments of the present disclosure, according to FIG. 6, the third connecting end of the cable may be but is not limited to a wiring terminal, which may be crimped at a bottom of the cable (i.e., the third connecting end), for example, a wiring terminal 1011 may be crimped at the bottom of the cable 101, a wiring terminal 1021 may be crimped at the bottom of the cable 102, and a wiring terminal 1031 may be crimped at the bottom of the cable 103. The six wiring terminals may correspond to the voltage phase distribution X+, X−, Z+, Z−, Y+, and Y−, respectively. The connection between the cable and the electrode can be achieved by crimping the hole position at one end of the electrode with the wiring terminal of the cable through a locking nut. The hole position at the other end of the electrode strip can also be connected to the current guide pillar through the locking nut, achieving the connection between the electrode strip and the current guide pillar. In some embodiments, by setting the first connecting end and a second connecting end at both ends of the electrode, a third connecting end on the cable, and the first connecting end and the second connecting end connecting with the third connecting end of the cable and the conductive pillar in the conductive pillar insulation device, respectively, the connection between the cable and the electrode and the connection as between the electrode and the conductive pillar can be achieved, which can facilitate the power supply component of the gradient coil to supply power to the gradient coil.

In some embodiments, the electrode insulation device and/or the magnetic resonance imaging system may be arranged with a first fixing component on the end surface of the magnet, and the first fixing component may be configured to fix the electrode insulation device on the end surface of the magnet. The fixing component may be but is not limited to bolts, nuts, pins, hooks, clamps, self-locking pressure plates, collars, straps, movable rings, or fixed rings. Specifically, the fixing component may be arranged on the end surface of the magnet of the electrode insulation device and the magnetic resonance imaging system, only on the electrode insulation device, or only on the end surface of the magnet. According to FIG. 2 and FIG. 5, screw holes may be arranged on the electrode insulation device, and a protruding cylinder may be welded on an outer wall of the main magnet end face, a center of the cylinder may be a threaded hole, by passing a screw through the screw hole on the electrode insulation device and the threaded hole on the magnet, the electrode insulation device 500 may be fixed on the end surface of the main magnet. The count of screw holes and cylinders may both be 4. The end surface of the magnet may be arranged with a clamp, a size of the clamp may be matched with the electrode insulation device, and the electrode insulation device may be fixed on the end surface of the main magnet 300 in a clamping manner. The electrode insulation device may be further arranged with a strap, and the strap may be configured to fix the electrode insulation device on the end surface of the main magnet 300. In some embodiments, by setting a first fixing member on the end surface of the main magnet of the electrode insulation device and/or magnetic resonance imaging system, the electrode insulation device may be fixed on the end surface of the magnet through the first fixing component, thereby fixing the power supply component of the gradient coil and solving the problem of cables being prone to breakage in the magnetic resonance imaging system.

In some embodiments, the power supply component of the gradient coil may include a second fixing component and the second portion may be configured to fix the cable on the end surface of the maim magnet. According to FIGS. 1-3 and 6, the power supply component of the gradient coil may also include a second fixing component 700 for fixing the cable on an outer wall of the end face of the main magnet. FIG. 7 is a schematic diagram illustrating a structure of a second fixing component according to some embodiments of the present disclosure. According to FIG. 7, the second fixing component 700 may include a first crimping plate 701 and a second crimping plate 702, a size of the first crimping plate 701 may be matched with a size of the second crimp plate 702 and the first crimping plate 701 and the second crimping plate 702 may be reserved with holes for the cable to pass through. For example, three holes may be reserved between the first crimping plate 701 and the second crimping plate 702 for three sets of cables to pass through. In some embodiments, additional holes may be provided on the second fixing component 700 configured to fix the second fixing component 700 on the end surface of the main magnet. For example, the second fixing component 700 may be arranged with two threaded holes to fix the second fixing component 700 on the end surface of the main magnet through a screw connection. By setting that the power supply component of the gradient coil further includes the second fixing component, the second fixing component may fix each set of cables on the end surface of the main magnet, which can solve the problem of the cable being prone to breakage in the magnetic resonance imaging system.

In some embodiments, the electrode insulation device may be arranged with a safety shield. FIG. 8 is a schematic diagram illustrating a structure of a safety shield of a second insulation device according to some embodiments of the present disclosure. As shown in FIG. 8, an upper portion of the second insulation device 500 may be arranged with a first safety shield 2012, and a lower portion of the second insulation device 500 may be arranged with a second safety shield 2013. Both the first safety shield 2012 and the second safety shield 2013 may be made of insulation material. By setting the safety shield on the second insulation device, the safety of the power supply component of the gradient coil can be improved and the risk of electric shock can be reduced.

In some embodiments, the power supply component 200 of the gradient coil may be oriented in any direction toward the end surface of the main magnet. Specifically, the power supply component 200 of the gradient coil may face towards a ceiling or ground direction on the end surface of the main magnet, and may also face towards a horizontal or other direction on the end surface of the main magnet. As shown in FIG. 1, when the power supply component of the gradient coil is arranged towards the ceiling direction on the end surface of the main magnet, the cable may be arranged at the ceiling. FIG. 9 is a schematic diagram illustrating a structure of a power supply assembly of a gradient coil facing the ground according to other embodiments of the present disclosure. As shown in FIG. 9, the power supply component of the gradient coil may be arranged towards the ground on the end surface of the main magnet, and the cable may be arranged on the ground. By orienting the power supply component of the gradient coil towards the end surface of the magnet in any direction, the cable may be flexibly arranged, which can improve the flexibility of using the power supply component of the gradient coil.

For those skilled in the art to have a deep understanding of the technical solution in the embodiments of the present disclosure, the following may be explained with specific examples.

Please refer to FIG. 1 and FIG. 2, the present disclosure provides a cable configured to supply power to a gradient coil in the magnetic resonance system, including a magnet system, a gradient system, and a cable 100 of the gradient coil, wherein:

The magnet system may include a main magnet 300 configured to generate a stable and uniform high magnetic field;

The gradient system may include a gradient coil 600 and a conductive pillar component that flows current to the gradient coil;

The cable of the gradient coil configured to supply power to gradient coil 600, including a conductive insulation device component, an electrode insulation device component, a cable 100, and a fixing component, wherein:

The conductive pillar insulation device component may be configured to lead out a considerable length of conductive pillar and connect the conductive pillar with the electrode insulation device component, wherein the conductive pillar and insulation material (e.g., epoxy resin) may poured and formed together;

The electrode insulation device component may be configured to connect the cable 100 to a bundle cable and the conductive insulation device component, thus achieving right angle turning and electrical connection of the cable on the aperture of the magnet. The electrode may be bent into a 90° shape and made of good conductor material and relatively soft material, which can withstand a certain amplitude of strength without fatigue fracture;

The cable 100 may be configured to supply power to the gradient coil;

The fixing component may be configured to fix the cable on the magnet.

According to the principles of electromagnetics, there are magnetic induction lines of certain strength distributed at the aperture of the magnet and the positions of two end bell mouths of the aperture of the magnet. If a bundled cable is directly connected, Lorentz force may be generated during vibration when passing through the current, which cannot achieve a good fixation. The electrode insulation device component 200 may fix the cable on the outer wall of the end surface of the magnet.

The gradient coil may have three sets of cables X+, X−, Y+, Y−, Z+, and Z−, which may correspond to six conductive pillars. According to FIG. 4, these three sets of voltage phase distributions may be X+, X−, Z+, Z−, Y+, and Y− from left to right. The three sets of conductive pillars may be fixed in a forming cavity and pour the conductive pillars into a well insulation material, such as epoxy resin adhesive for solidifying and forming, to match the gradient coil in a tubular shape, it is generally poured into a semicircle shape, wherein 401 represents six conductive pillars, 400 represents the insulation device component of the conductive pillar after solidification which has good insulation, and 403 represents the cooling pipe, before pouring, it is arranged around the conductive pillar to achieve the purpose of cooling the conductive pillar; in order to have a good connection during the pouring operation with the gradient coil in a later stage, the shape of the cooling pipe 403 may be designed as an arc-ladder shape, with a relatively large volume at the rear of the cooling pipe 403 and a relatively small volume at the front of the cooling pipe 403, which can achieve good axial fixation.

A manufacturing manner of the gradient coil 600 may include: pouring the spiral coil made of wires into a tube shape using epoxy resin, fixing a pre-poured conductive pillar insulation device on the gradient coil forming mold, and pouring the pre-poured conductive pillar insulation device with the gradient coil to obtain an integrated conductive pillar insulation device and the gradient coil 600, and integrating the conductive pillar insulation device and the gradient coil 600 as a whole.

Traditional cables cannot achieve good turns at right angles due to a large bending radius or being limited by space, according to FIG. 5, the electrode may be poured into a forming cavity, which may correspond to positions and dimensions of electrical phases X+, X−, Z+, Z−, Y+, and Y− in FIG. 4, wherein 503 represents X+, 504 represents X−, 505 represents Z+, 506 represents Z−, 507 represents Y+, and 508 represents Y−. The six electrodes may be pre-bent into a 90° shape, one end of the six electrodes may be poured into a whole, and a length of another bent end may be calculated to be controlled within a range of 100-150 mm, which can achieve good vibration absorption effect.

According to FIG. 5, hole positions may be reserved at both ends of each electrode in the electrode insulation device component, and hole positions may be fixed and connected with locking nuts during integration, the electrode insulation device component may be fixed as a whole on the outer wall of the end surface of magnet through locking nuts, and may be fixed with 4 screw holes. Correspondingly, 4 protruding cylinders may be welded on the outer wall of the end surface of the magnet, and the center of the cylinder may be a threaded hole to achieve the threaded connection.

According to FIG. 8, the electrode insulation device component and the conductive pillar insulation device component may be connected with locking nuts. At the same time, in order to improve safety and prevent the risk of electric shock, safety shields 2012 and 2013 made of insulation material may be arranged at a certain position below the electrode insulation device component.

According to FIG. 6, a lower portion of cable 100 may be crimped with wiring terminals, wherein 101, 102, and 103 represent three sets of cables, and lower forks may be crimped with wiring terminals X+, X−, Z+, Z−, Y+, Y− represented by 1011, 1021, and 1031, and then crimped together with the electrode insulation device component using a locking nut.

According to FIG. 7, the fixing component may fix the cable on the outer wall of the end surface of the magnet, wherein 701 and 702 represent the crimping plate, respectively. After pressing the cable tightly, the whole may be fixed.

According to FIG. 9, the cable of the gradient coil may be arranged in a vertical 90° direction (i.e., the cable faces towards the ceiling) or in a rotating downward direction (i.e., the cable faces towards the ground), indicating that the cable may be arranged on a roof ceiling or routed on the ground, which can improve flexibility in cable installation.

The cable component configured to supply power the gradient coil in the magnetic resonance system may be formed by pouring the electrodes and conductive pillars onto the insulation device, integrating the insulation device into the magnetic resonance system, which allows the current to pass through the cable, the electrodes, and conductive pillars in sequence to power the gradient coil, since power supply lines of the gradient coil are fixed by the electrode insulation device and the conductive insulation device, the problem of cable components being prone to breakage in the magnetic resonance imaging system can be solved.

According to FIG. 1, the present disclosure provides a magnetic resonance imaging system, including a main magnet 300, a gradient coil 600, a cable 100, and a power supply component of the gradient coil. The power supply component of the gradient coil may include an electrode insulation device and a conductive insulation device matched with the gradient coil 600 of the magnetic resonance imaging system; the electrode insulation device may be arranged with an electrode, and the conductive pillar insulation device may be arranged with a conductive pillar; the electrode may be connected with the cable 100 of the magnetic resonance imaging system and the conductive in the conductive insulation device, respectively, to supply power to the gradient coil 600.

Specifically, the gradient coil 600 may be arranged inside the main magnet 300, and the power supply component of the gradient coil may include an electrode insulation device and a conductive pillar insulation device. The electrode insulation device may be arranged with an electrode, the conductive pillar insulation device may be arranged with a conductive pillar, and the electrodes in the electrode insulation device may be connected with the cable 100 and the conductive pillar in the conductive pillar insulation device, respectively. The current may flow through the cable 100, the electrodes in the electrode insulation device, and the conductive pillar in the conductive pillar insulation device to flow to the gradient coil 600 in the main magnet 300 to supply power to the gradient coil 600 in the main magnet 300.

The magnetic resonance imaging system may supply power to the gradient coil 600 by setting an electrode in the electrode insulation device and a conductive pillar in the conductive pillar insulation device, connecting the electrode to the cable 100 and the conductive pillar, respectively, which allows the current to pass through the cable 100, the electrode in the electrode insulation device, and the conductive pillar in the conductive pillar insulation device in sequence to supply power to the gradient coil 600, due to the electrical connection component of gradient coil 600 being fixed through the electrode insulation device and conductive insulation device, the problem of cables being prone to breakage in the magnetic resonance imaging system can be solved.

In some embodiments, the imaging device 110 may send the acquired image to the processing device 120. The imaging device 110 may receive instructions sent by doctors through the terminal 140, and perform related operations based on the instructions, such as scanning and imaging. In some embodiments, the imaging device 110 may exchange data and/or information with other components in the system 100 (e.g., the processing device 120, the storage device 130, the terminal 140) through the network 150. In some embodiments, the imaging device 110 may be directly connected to other components in the system 100. In some embodiments, the imaging device 110 may include one or more components (e.g., the processing device 120, the storage device 130) in the system 100.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A power supply assembly of a gradient coil, comprising:
a first insulation device (400), wherein the first insulation device (400) is located within a scanning channel (301) of a main magnet (300) of a magnetic resonance imaging (MRI) system, the first insulation device (400) is arranged with a first conductive unit (401), the first conductive unit (401) is a rigid conductor, a first end of the first conductive unit (401) is electrically connected with the gradient coil (600) of the MRI system, and a second end of the first conductive unit (401) is electrically connected with a cable (100) of the MRI system.

2. The power supply assembly of the gradient coil of claim 1, wherein the first insulation device (400) further includes an insulated fixing module (402) that wraps the first conductive unit (401).

3. The power supply assembly of the gradient coil of claim 2, wherein the insulated fixing module (402) and the first conductive unit (401) are integrally formed.

4. The power supply assembly of the gradient coil of claim 1, wherein the first insulation device (400) and the gradient coil (600) are integrally formed.

5. The power supply assembly of the gradient coil of claim 4, wherein the first insulation device (400) and the gradient coil (600) are integrally formed using an integrated forming device including:
a gradient coil forming mold (803) configured to accommodate the gradient coil (600) and a portion of the first insulation device (400) connecting with the gradient coil (600);
a first supporting component (802) configured to support the first insulation device (400), the first supporting component (802) being connected with the gradient coil forming mold (803); and
a second supporting component (801) configured to support the first insulation device (400), the second supporting component (801) being connected with the first insulation device (400) and the first supporting component (802).

6. The power supply assembly of the gradient coil of claim 5, wherein
a first end of the first supporting component (802) is connected with an end surface of the gradient coil forming mold (803) closed to first insulation device (400);
the second supporting component (801) is connected with a second end of the first supporting component (802) and a second end of the first insulation device (400), the first end of the first supporting component (802) being opposite to the second end of the first supporting component (802), and the second end of the first insulation device (400) corresponding to the second end of the first conductive unit (401).

7. The power supply assembly of the gradient coil of claim 5, wherein the second supporting component (801) includes at least one groove configured to accommodate the first conductive unit (401).

8. The power supply assembly of the gradient coil of claim 2, wherein the first insulation device (400) is arranged with a cooling pipe (403) configured to cool the first conductive unit (401).

9. The power supply assembly of the gradient coil of claim 8, wherein the insulated fixing module (402) wraps the cooling pipe (403).

10. The power supply assembly of the gradient coil of claim 1, wherein the first insulation device (400) includes an arc-shaped structure, and a radian of the arc-shaped structure is matched with a radian of the gradient coil (600).

11. The power supply assembly of the gradient coil of claim 1, wherein
a length of the first insulation device (400) along an axial direction of the MRI system is less than a target distance;
along the axis direction, the gradient coil (600) includes a first end, and the main magnet (300) of the MRI system includes a second end;
the first end of the gradient coil (600) corresponds to the second end of the main magnet (300) of the MRI system; and
the target distance is a distance between the first end of the gradient coil (600) and the second end of the main magnet (300) of the MRI system along the axis direction.

12. The power supply assembly of the gradient coil of claim 11, wherein a ratio of the length of the first insulation device (400) to the target distance is 0.5-0.8.

13. The power supply assembly of the gradient coil of claim 1, wherein
along an axial direction of the MRI system, the first insulation device (400) includes a first end and a second end;
the first end of the first insulation device (400) corresponds to the first end of the first conductive unit (401), and the second end of the first insulation device (400) corresponds to the second end of the first conductive unit (401); and
a size of a cross section of the first end of the first insulation device (400) is smaller than a size of a cross section of the second end of the first insulation device (400).

14. The power supply assembly of the gradient coil of claim 1, further comprising a second insulation device (500), wherein
the second insulation device (500) is fixed on an end surface of a main magnet (300) of the MRI system, the second insulation device (500) is arranged with a second conductive unit (503, 504, 505, 506, 507, 508),
a first end of the second conductive unit (503, 504, 505, 506, 507, 508) is electrically connected with the cable (100) of the MRI system, and
a second end of the second conductive unit (503, 504, 505, 506, 507, 508) is electrically connected with the second end of the first conductive unit (401).

15. (canceled)

16. (canceled)

17. The power supply assembly of the gradient coil of claim 14, wherein
the second conductive unit (503, 504, 505, 506, 507, 508) includes a first portion and a second portion,
the first portion extends into the scanning channel (301) of the main magnet (300) of the MRI system and is connected with the first insulation device (400),
the second portion is fixed on the end surface of the main magnet (300) of the MRI system, and
there is a preset angle between the first portion and the second portion.

18. (canceled)

19. The power supply assembly of the gradient coil of claim 17, wherein a width of the second conductive unit (503, 504, 505, 506, 507, 508) is negatively correlated with a length of the second portion.

20. (canceled)

21. (canceled)

22. The power supply assembly of the gradient coil of claim 1, further comprising a connecting component (804), wherein the connecting component (804) is connected with the first end of the first conductive unit (401) and is configured to prevent the first end of the first conductive unit (401) from being separated from the gradient coil (600).

23. The power supply assembly of the gradient coil of claim 1, wherein the power supply assembly (200) is configured to be disposed in any radial direction of the gradient coil (600) of the MRI system.

24. A magnetic resonance imaging (MRI) system, comprising:
a main magnet (300);
a gradient coil (600);
a power supply assembly (200) of the gradient coil (600); and
a cable (100); the cable (100) configured to supply power to the gradient coil through the power supply assembly of the gradient coil;
wherein the power supply assembly (200) of the gradient coil (600) includes a first insulation device (400), the first insulation device (400) is located within a scanning channel (301) of the main magnet (300), the first insulation device (400) is arranged with a first conductive unit (401), the first conductive unit (401) is a rigid conductor, a first end of the first conductive unit (401) is electrically connected with the gradient coil (600), and a second end of the first conductive unit (401) is electrically connected with the cable (100).

25. A power supply assembly of a gradient coil, comprising:
an electrode insulation device and a conductive pillar insulation device matched with a gradient coil (600) of a magnetic resonance imaging (MRI) system;
wherein the electrode insulation device is arranged with an electrode, the conductive pillar insulation device is arranged with a conductive pillar, and the electrode is connected with the cable (100) and the conductive pillar to supply power for the gradient coil (600).

26-33. (canceled)
